(19) United States
(12) Patent Application Publication
(10) Pub. No.: US 2016/0096000 A1
(43) Pub. Date: Apr. 7, 2016

(54) MICROPUNCTURE VASCULAR ACCESS SET

(71) Applicant: Auxilium Device Research, LLC, Grand Rapids, MI (US)

(72) Inventor: Jihad A. Mustapha, Ada, MI (US)

(73) Assignee: Auxilium Device Research, LLC

(21) Appl. No.: 14/503,653

(22) Filed: Oct. 1, 2014

(51) Int. Cl. A61M 25/00; A61M 25/01; A61B 17/34; A61M 25/06

(52) U.S. Cl. CPC A61M 25/007; A61M 25/0662; A61M 25/0102; A61B 17/3415; A61B 2017/3454

(57) ABSTRACT

A vascular access device includes a sheath body defining an outer surface and a lumen extending between a first end of the sheath body and a second end of the sheath body. The sheath body has a length of at most 7 cm. Further, the sheath body has a vessel opening defined through the first end in communication with the lumen and an access opening defined through the second end in communication with the lumen. A plurality of perfusion windows extend between and are open to the lumen and the outer surface of the sheath body, each of the plurality of perfusion windows being located within about 3 cm of the first end of the sheath body.

### FIELD OF THE INVENTION

The present invention generally relates to a vascular access sheath and related system. In particular, the access sheath includes a plurality of perfusion windows to promote blood flow therethrough.

### BACKGROUND OF THE INVENTION

Various procedures exist that are aimed at the treatment of damaged or diseased portions of the human vasculature. Such procedures may include peripheral angioplasty, and may further include atherectomy, drug delivery, or peripheral stenting. These, and other related procedures are carried out using catheterized tools positioned within the interior (lumen) of the damaged blood vessel, which may be a vein or artery, and accordingly, require percutaneous access to the lumen. Such access may be gained by placing a sheath through the skin and underlying tissue (if present) of the patient and further through the close wall of the vessel into the lumen. The sheath may function to maintain the thusly-created openings in the patient and to provide a path for the insertion of the various catheters to be used in the procedure.

When such procedures are carried out in the legs or arms of a patient, the diseased portion of the vessel may be accessed from either an antegrade direction (i.e. from more proximal to the heart) or in a retrograde direction (i.e. from farther away from the heart). In some circumstances, retrograde access, such as through an opening created near the foot or hand (e.g. ankle or wrist) of the patient) may be preferable, however, the veins or arteries of the patient are naturally more narrow in such locations to the extent that a sheath may block the flow of blood to locations distal of the access point. As a result, only up to about five minutes of operation time may be available before the removal of the sheath and the other instruments inserted therethrough is required at frequent intervals.

Given that the types of procedures described above may take one or more hours, removal of a sheath every five minutes to allow for blood to flow to the foot or hand, for example, may add significantly to the amount of time need to perform the procedure. Accordingly, further advances in access sheaths may be desired.

### SUMMARY OF THE INVENTION

According to one aspect of the present invention, a vascular access device includes a sheath body defining an outer surface and a lumen extending between a first end of the sheath body and a second end of the sheath body. The sheath body has a length of at most 7 cm. Further, the sheath body has a vessel opening defined through the first end in communication with the lumen and an access opening defined through the second end in communication with the lumen. A plurality of perfusion windows extend between and are open to the lumen and the outer surface of the sheath body, each of the plurality of perfusion windows being located within about 3 cm of the first end of the sheath body. In an example, the plurality of perfusion windows may include exactly three perfusion windows. Additionally or alternatively, the perfusion windows may be distributed along a section of the length of the sheath body and around a circumference of the outer surface of the sheath body in a spiral pattern. Each of the plurality of perfusion windows may have a diameter of between about 8/1000 in and about 12/1000 in.

According to another aspect of the present invention, a vascular access system includes an access sheath including a body extending between an access end and a subcutaneous end and defining a lumen therethrough open between the access end and the subcutaneous end. An outer surface of the lumen extends between the access end and the subcutaneous end coaxially with the lumen, and a plurality of perfusion windows extend between and are open to the lumen and the outer surface of the sheath body. The system further includes an introducer assemblable within the lumen of the access sheath through the access end thereof so as to extend outwardly through the subcutaneous end.

According to another aspect of the present invention, a vascular access sheath includes a sheath body defining an outer surface and an interior lumen. The sheath body defines a first end and a second end opposite the first end. The outer surface and the interior lumen extend coaxially between the first end and the second end, and a plurality of perfusion windows extend between and are open to the lumen and the outer surface of the sheath body within about 3 cm of the first end. The lumen defines an access path between an access opening through the second end and a subcutaneous opening through the first end. The lumen further defines a perfusion path between the subcutaneous opening and the perfusion windows. In an example, the perfusion path may be generally defined around at least a portion of the access path.

These and other aspects, objects, and features of the present invention will be understood and appreciated by those skilled in the art upon studying the following specification, claims, and appended drawings.

### BRIEF DESCRIPTION OF THE DRAWINGS

In the drawings:

FIG. 1 is a side elevational view of assembled components of vascular access system according to an aspect of the present disclosure;

FIG. 2 is a detail view of a portion of the access system of FIG. 1;

FIG. 3 is a cross-sectional view of the assembled components of the vascular access system take along line III-III of FIG. 2;

FIG. 4 is a plan view of the assembled components of FIG. 1 inserted into a portion of the vasculature of a patient;

FIG. 5 is a partial lateral cross-sectional view of a portion of the assembled components of the vascular access system;

FIG. 6 is a detail view of a sheath and guidewire of the vascular access system within the lumen of a blood vessel;

FIG. 7 is the detail view of FIG. 6 further illustrating an example of blood flow within and through a portion of the sheath;

FIG. 8 is a detail view of the sheath and guidewire of the vascular access system with a catheter further assembled therewith, illustrating an example of blood flow within and through a portion of the sheath and around the catheter;

FIG. 9 is the detail view of FIG. 6 further illustrating an example of a delivery flow within and through a portion of the sheath; and

FIG. 10 is a detail view of a portion of a sheath for a vascular access system according to another aspect of the present disclosure.

### DETAILED DESCRIPTION OF THE PREFERRED EMBODIMENTS

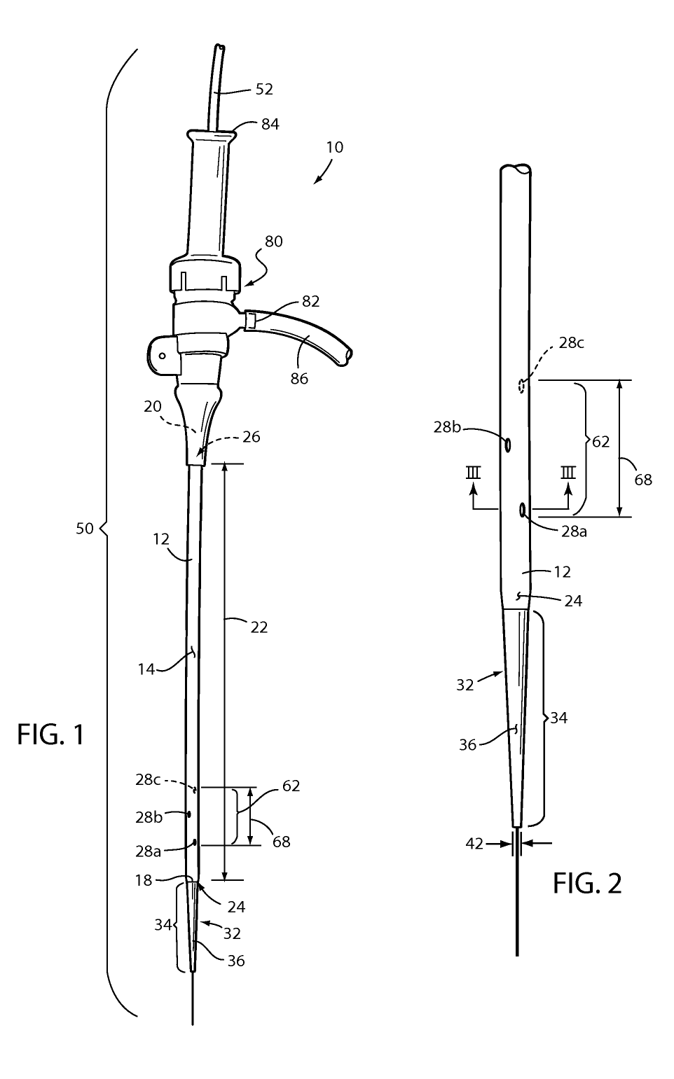

For purposes of description herein, the terms “upper,” “lower,” “right,” “left,” “rear,” “front,” “vertical,” “horizontal,” “interior,” “exterior,” and derivatives thereof shall relate to the invention as oriented in FIG. 1. However, it is to be understood that the invention may assume various alternative orientations, except where expressly specified to the contrary. It is also to be understood that the specific devices and processes illustrated in the attached drawing, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise. Additionally, unless otherwise specified, it is to be understood that discussion of a particular feature of component extending in or along a given direction or the like does not mean that the feature or component follows a straight line or axis in such a direction or that it only extends in such direction or on such a plane without other directional components or deviations, unless otherwise specified.

Referring to FIG. 1, reference numeral 10 generally designates a vascular access device. Vascular access device 10 includes a sheath body 12 defining an outer surface 14 and a lumen 16 extending between a first end 18 of the sheath body 12 and second end 20 of the sheath body 12. Sheath body 12 has a length of at most 7 cm, and has a vessel opening 24 defined through first end 18 in communication with lumen 16 and an access opening 26 defined through second end 20 in communication with the lumen 16. A plurality of perfusion windows 28a, 28b, and 28c extend between and are open to lumen 16 and outer surface 14 of sheath body 12.

As shown in FIGS. 1-3, the vascular access device 10 can be a part of a vascular access system 30 that can further include an introducer 32 that can be assembled within sheath 12, such as by passing through access opening 26 and through lumen 16 such that tip 34 thereof extends outwardly beyond first end 18 of sheath body 12 and out of vessel opening 24. As shown in FIGS. 1 and 2, introducer 32 can be configured such that the tip portion 34 thereof is positioned generally completely outside of sheath body beyond second end 20 thereof when the introducer 32 is assembled with the sheath body 12. The tip portion 34 of introducer 32 can be distinguished from remaining portions of introducer 32 by the tapering of the outer surface 36 of introducer 32, which can take place entirely within tip portion 34 of introducer 32. As such, surface 36 of introducer 32 can be generally uniform outside of tip portion 34 and can have a diameter 38 that can generally match an inner diameter 40 of lumen 16 (while being generally undersized by an acceptable amount necessary for the introducer 32 to be slid within lumen 16 without the need for excessive force, including accounting for necessary material tolerances). Surface 36 of introducer 32 can then taper within tip portion 34 from diameter 38 down to a tip diameter 42 that is less than diameter 38.

As shown in FIG. 2, the outer surface 14 of sheath body 12 can also taper within a tapered region 44, positioned adjacent first end 18 at a rate and over a predetermined portion of sheath body 12 such that the taper within tapered area 44 of sheath body 12 generally matches (i.e. is within about 15 degrees of) the taper within tip portion 34 of introducer 32. This can result in a generally smooth or seamless transition between the sheath body 12 and the introducer 32. In one example, interior diameter 40 of lumen 16 can be about 2.9 fr. As discussed above, diameter 38 of introducer 32 can be generally equal to, or somewhat undersized relative to interior diameter 40 of lumen 16, such that in a particular embodiment where interior diameter 40 of lumen 16 is about 2.9 fr, diameter 38 can be about 2.9 fr or can be about somewhat less than 2.9 fr, such as between about 1% and about 10% less. Diameter 42 at the end of tip area 34 of introducer 32 can be about 0.021 inches. Further, tip area 34 of introducer 32 can have a length of at least about 2 cm.

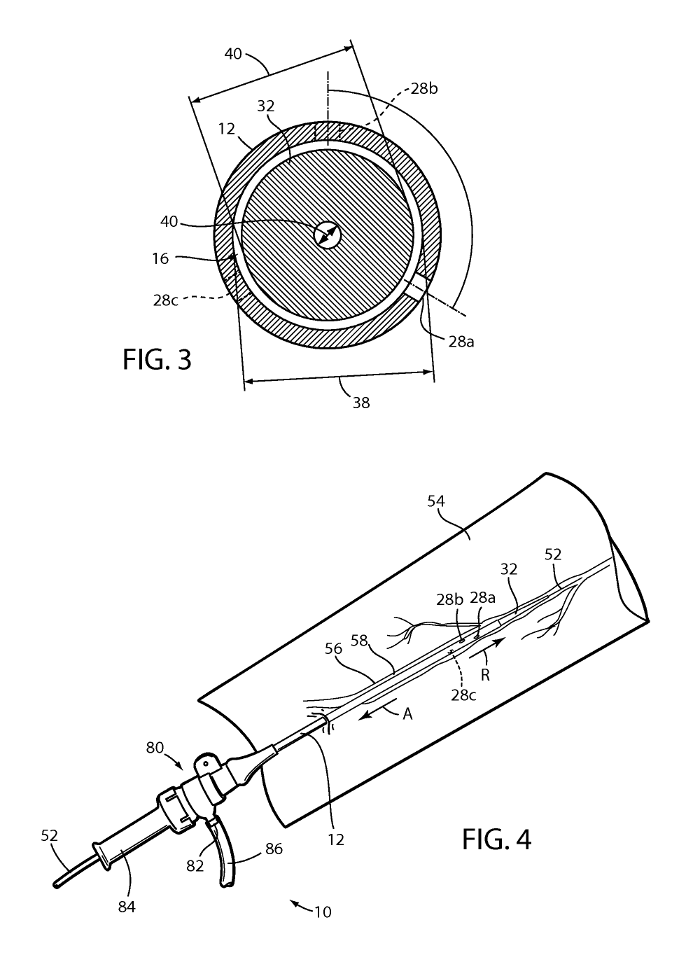

As shown in the cross-sectional view of FIG. 3, introducer 32 can define a guidewire path 48 therein that extends through the entirety thereof in a coaxial manner with lumen 16, when introducer 32 is assembled within sheath body 12. In an example, the inner diameter 50 of guidewire path 48 can be substantially equal to the diameter 42 of the end of tip 34 of introducer 32, such that outer surface 36 of introducer 32 extends generally away from guidewire path 48 on the outside thereof. Accordingly, diameter 50 of guidewire path 48 can be about 0.021 inches (+/−10%) so as to be capable of closely receiving a 21k guidewire such as a guidewire 52, therethrough, as illustrated in FIGS. 1 and 2, showing guidewire 52 as a part of system 30.

The assembly of system 30 as shown in FIGS. 1 and 2, including the assembly of introducer 32 within sheath body 12 and the assembly of guidewire 52 through the guidewire path 48 within introducer, 32 can be used to position at least a portion of sheath body 12 within the vasculature of a patient. Such positioning can allow a user of device 10, such as a surgeon or the like, to gain access to the patient's vasculature using device 10, as discussed further below. In use, the guidewire 52 can be pressed into the skin of a patient, such as a human patient, under the aid of x-ray guidance or the like, such that the guidewire 52 passes through the skin and into a selected vessel 56 (illustrated as an artery, but which can include either a vein or an artery) within a particular limb 54 of the patient. The guidewire 52 can then be extended within an interior, or lumen 58, of the selected vessel 56 sufficient to either reach an operative area within the lumen 58 or at least long enough to establish a direction within the lumen 58 in which device 10 will be inserted.

The assembled device 10, including sheath 12 and introducer 32, can then be assembled over guidewire 52, by positioning introducer 32, with sheath body 12 thereover, onto guidewire 52 by inserting an end of guidewire 52 outside of the limb 54 within guidewire path 48 through introducer tip 34. The assembled sheath 12 and introducer 32 can then be slid over wire until tip 34 is in contact with the skin surrounding guidewire 52. The assembled sheath body 12 and introducer 32 can then be further forced into limb 54 such the taper of introducer tip 34 forms an opening within the skin surrounding the inserted guidewire 52, which continues through the vessel wall, such that introducer 32 enters the lumen 58 therein. Continued force on the assembled sheath 12 and introducer 32 can, thusly, move at least a portion of sheath body 12 into the lumen 58, as shown in FIG. 4.

To help facilitate insertion of sheath body 12 into lumen 58 of a desired vessel 56, introducer 32 can possess appropriate strength and/or rigidity to properly traverse the skin and vessel 56, thereby promoting effective access. Such appropriate tensile strength can ensure that introducer 32 is rigid enough to appropriately translate the force applied to the combined sheath 12 and introducer 32 to the skin and vessel 58 such that an appropriate level of force thereon effectively allows introducer 32, and subsequently sheath 12 to enter vessel 56 without coiling or kinking within vessel 56. Appropriate strength of introducer 32 can also reduce the likelihood that introducer 32 is of an excessive rigidity that would place the contralateral side of the vessel 56 at risk for endothelial or arterial wall trauma, resulting through tip 34 traversing through a potentially calcified vessel 56 in attempt to access the lumen 58 therein. In an example, introducer 32 can be of a polymeric material, such as polyethylene, polypropylene, or the like. Such material may further be coated with silicone to promote insertability thereof through the skin and, in combination with the tapered shape of tip 34, expansion of the puncture created by guidewire 52. Further, the material comprising introducer 32 can include radiolucent properties, such as by the addition of particular additives to the base polymeric material thereof, thereby facilitating the visibility thereof by X-ray guidance or the like. In accordance with the above, the assembled sheath body 12 and introducer 32 can be positioned at a desired location within lumen 58 of vessel 56 such that the first end 18 of the sheath body 12 is within lumen 58 and at a distance from the insertion point of up to approximately the length 22 of sheath 12.

As shown in FIG. 4, as well as in FIG. 6, introducer 32 can be subsequently removed from sheath 12 by drawing introducer 32 from out of the vessel opening 24 and off of the aforementioned end of the guide wire 52 positioned outside of the limb 54. Accordingly, sheath 12 can remain in place within lumen 58 of vessel 56 to provide an access pathway through the arterial lumen that can be used for access by a catheterized tool (as discussed further below) to a diseased portion of the vessel 56 beyond the first end 18 of sheath 12. In this respect, sheath body 12 may be made of a material, such as a polymeric material including polyethylene, polypropylene, or the like, having a stiffness sufficient to maintain the integrity of the access pathway thus provided, while allowing flexibility to extend into the lumen 58 of the vessel 56 without causing damage to the vessel 56 or the skin of the patient surrounding sheath body 12. As shown in FIG. 4, access to the lumen 58 of vessel 56 by sheath body 12 can be gained in retrograde direction R, which is against the flow of blood within vessel 56. In the illustration of FIG. 4, sheath 12 is shown inserted into a vessel 56 that is an artery and the retrograde direction R is in a proximal direction (toward the heart). In situations where sheath 12 is used to gain access to a vessel 56 in the form of a vein, the retrograde direction R would be in a distal direction (away from the heart). Access in such a retrograde direction R can allow for a retrograde approach beyond sheath 12 to a damaged portion of the vessel 56 such as an over-calcified portion thereof, from an insertion point 60 in antegrade direction A from the damaged portion of the vessel 56.

In an example, limb 54 can be the leg of a patient, and insertion point 60 can be adjacent the foot of the patient, such as in the general area of the ankle or the like. In another example, limb 54 can be an arm of the patient and insertion point 60 can be adjacent the hand or wrist of the patient. Various repair procedures can be carried out using a tool (discussed below) positioned within vessel 56 by accessing the lumen thereof through sheath 12, such as stent installation within lumen 58, drug administration, balloon catheterization, atherectomy, or the like.

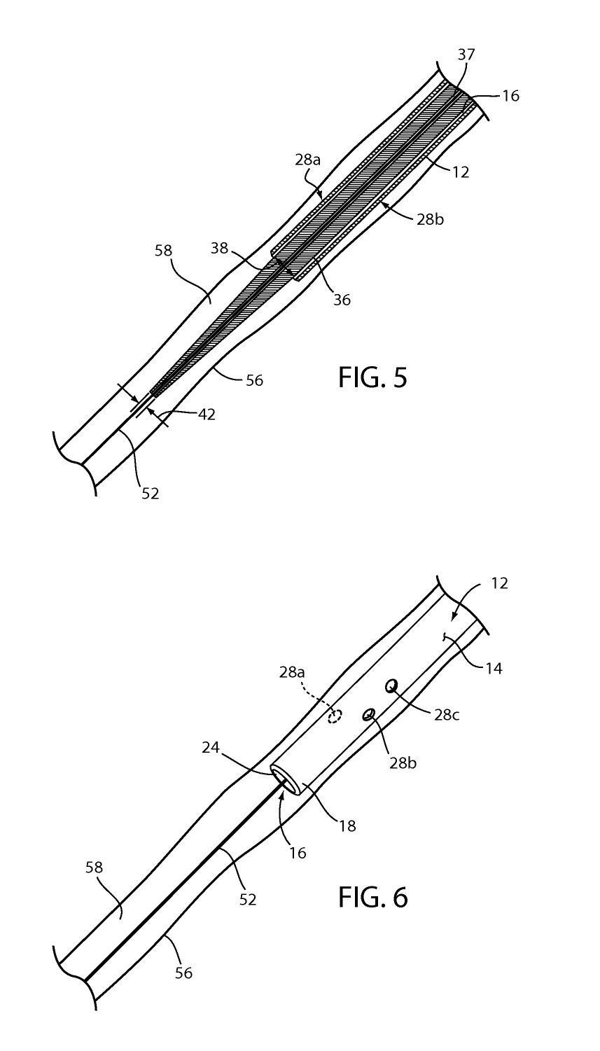

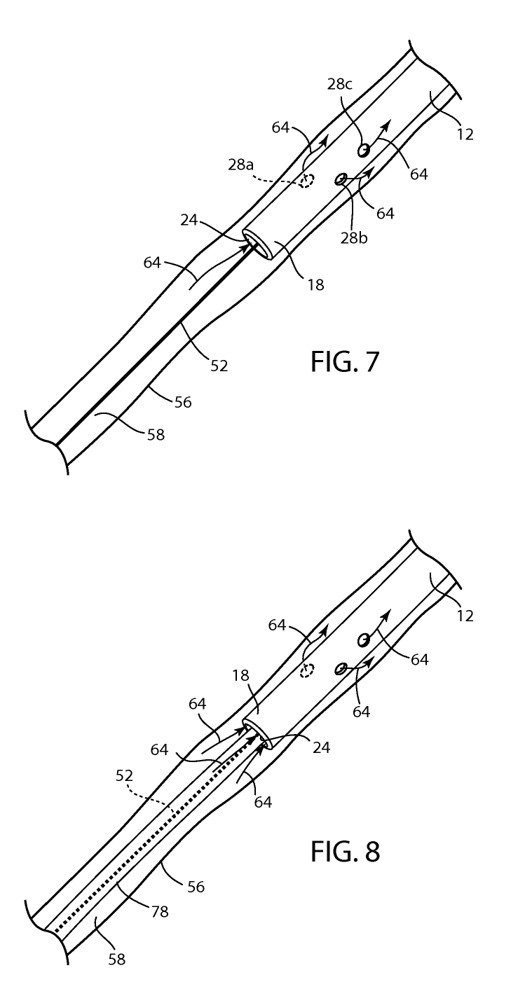

As shown in FIGS. 3-9, sheath 12 includes various features capable of reducing ischemia to parts of limb 54 in the antegrade direction A from sheath 12. As shown in FIG. 6, perfusion windows 28a, 28b, and 28c, which extend through sheath 12 between outer surface 14 and lumen 16, are positioned within an area 62 of sheath 12. In general, perfusion windows 28 can allow for the maintenance of blood flow through and around sheath 12 within the arterial lumen 58 to locations distal (i.e. in the antegrade direction A) from sheath 12. For example, as shown in FIG. 7, blood can enter vessel opening 24 from up stream of sheath 12 so as to flow into lumen 16 of sheath 12, where such blood can flow through a portion of sheath 12 within lumen 16 before exiting through a downstream perfusion window 28a, 28b, or 28c. As illustrated, a perfusion path 64, or a series of perfusion paths 64 are thusly available within a portion of sheath 12. Such a perfusion path 64 may not necessarily be separately defined within sheath 12 but may exist, in a general manner, between vessel opening 24 and one or more of the perfusion windows 28a, 28b, and 28c.

By allowing blood flow through a portion of sheath 12, along a perfusion path 64 or perfusion paths 64, the blockage of blood flow in the antegrade direction A through lumen 58 by sheath 12 is reduced, at least to an extent to where sheath 12 can be left in place within lumen 58 over a time longer than would be permitted without the presence of perfusion windows 28 within sheath 12, without presenting a substantial risk for ischemia to the portions of limb 54 downstream of sheath 12. In an example, a variation of sheath 12 without perfusion windows 28 may provide only up to about 5 minutes of operation time before the removal of such a sheath is required. Given that the types of procedures performed using a sheath 12 of such a type may take one or more hours, removal of a sheath every 5 minutes to allow for blood to flow to locations of lumen 54 downstream of the insertion point may add significantly to the amount of time need to perform a procedure using a sheath 12 without perfusion windows 28. In another example, a sheath 12 with perfusion windows 28, as described further herein, may allow for sheath 12 to be left within lumen 58 for the entire duration of a procedure, thereby greatly reducing the overall time needed to complete such a procedure.

As further shown in FIG. 3 and FIGS. 5-7, perfusion windows 28 can be incorporated into sheath 12 such that exactly three perfusion windows 28a, 28b, and 28c are present. Such perfusion windows 28a, 28b, 28c can be arranged in a spiral pattern along sheath body 12. The perfusion windows can be distributed along an axial length of sheath body 12 based on clinical need. In an example, the three perfusion windows 28a, 28b, and 28c can be evenly spaced apart along length 22 of sheath body 12. In another example, perfusion windows 28a, 28b, and 28c can be distributed along sheath body 12 in an uneven manner such as by having windows 28a and 28b positioned toward first end 18 with window 28c positioned either toward the middle of sheath body 12 or toward second end 20. In another example, perfusion window 28a can be positioned toward the middle of sheath body 12 with perfusion window 28c positioned toward the second end 20 of sheath body 12 and perfusion window 28b positioned either toward perfusion window 28a or perfusion window 28c. Other distributions of perfusion windows 28a, 28b, and 28c are possible based on similar schemes, including arrangements having more than three perfusion windows 28.

In another example, as shown in FIG. 2, perfusion windows 28a, 28b, and 28c can be positioned within window area 62 so as to all be positioned within, for example about 3 cm of first end 18 of sheath 12. Perfusion windows 28a, 28b, and 28c in such an arrangement can be distributed within a window area 62 that is between about 1 cm and 2 cm in length, and can further be evenly spaced along such a length 68, although various uneven distributions are possible. In one example, perfusion windows 28a and 28b, as well as 28b and 28c can be positioned at a distance 72 from each other of between 5 mm and about 10 mm. In other examples, window are 62 can be located toward the middle of sheath body 12, such that perfusion windows 28a, 28b, and 28c are located, for example, within about 2.5 cm and 4.5 cm from first end 18, or toward second end of sheath body 12, such that perfusion windows 28a, 28b, and 28c are located at least 5 cm from first end 18. Other locations for window are 62 and lengths thereof are possible.

In any of the above-described distributions of perfusion windows 28a, 28b, and 28c along length 22 of sheath body 12, discussed above, perfusion windows 28a, 28b, and 28c can further be evenly distributed axially around sheath body 12, as shown in FIG. 3, in which each of perfusion windows 28a, 28b, and 28c are successively positioned about 120 degrees from each other about an access of lumen 16. Such spiral positioning of perfusion windows 28a, 28b, and 28c can allow for generally uniform perfusion of blood along perfusion path 64 and can increase a likelihood that, should one perfusion window 28b positioned against the wall of artery 56, the remaining perfusion windows 28 will be available for blood to flow therethrough along alternative perfusion paths 64 in an amount sufficient to reduce or alleviate substantial risk of ischemia.

Each of the perfusion windows 28a, 28b, and 28c can have a diameter 76 of between about 8/1000 inches and about 12/1000 inches, each being generally uniform to within about 10% of each other. Such sizing of perfusion windows 28a, 28b, 28c can allow for appropriate distal perfusion along perfusion paths 64 without causing substantial damage to the cellular contents (i.e. hemolysis), and can further maintain generally optimal pressure within sheath 12 to promote outward flow and subsequent distal perfusion of blood out of perfusion windows 28. The incorporation of three perfusion windows 28 can provide such benefits without significantly weakening sheath 12 or increasing the possibility of sheath 12 folding upon itself by way of coiling or kinking. Further, the positioning of perfusion window are 62 within, for example for about 3 cm of first end 18 of sheath 12 can further help maintain the structural integrity of sheath 12.

As further shown in FIG. 8, an access path 66 can also be provided by lumen 18 of sheath 12. Such a path can be present as additional volume extending axially within lumen 18 in addition to the volume needed therein to achieve a desired perfusion flow along perfusion paths 64, for example. A catheter 78, such as a catheterized device, which can further be included in system 30, of an appropriate size may extend through such a volume long access path 66 such that perfusion paths 64 extend therearound. As illustrated, catheter 78 can be extended through lumen 16 and over guidewire 52, which may be in place within sheath 12 and further extended from out of vessel opening 24 and away from sheath to the location at which the procedure is to be conducted, in some examples can be at least 10 cm retrograde of first end 18 of sheath 12. As illustrated, catheter 78 can be undersized relative to lumen such that perfusion paths 64 remain present between areas of vessel opening 24 between the wall of lumen 16 and catheter 78 and perfusion windows 28a, 28b, and 28c. In this manner, the perfusion path 64 or perfusion paths 64 are generally defined around at least a portion of the access path 66.

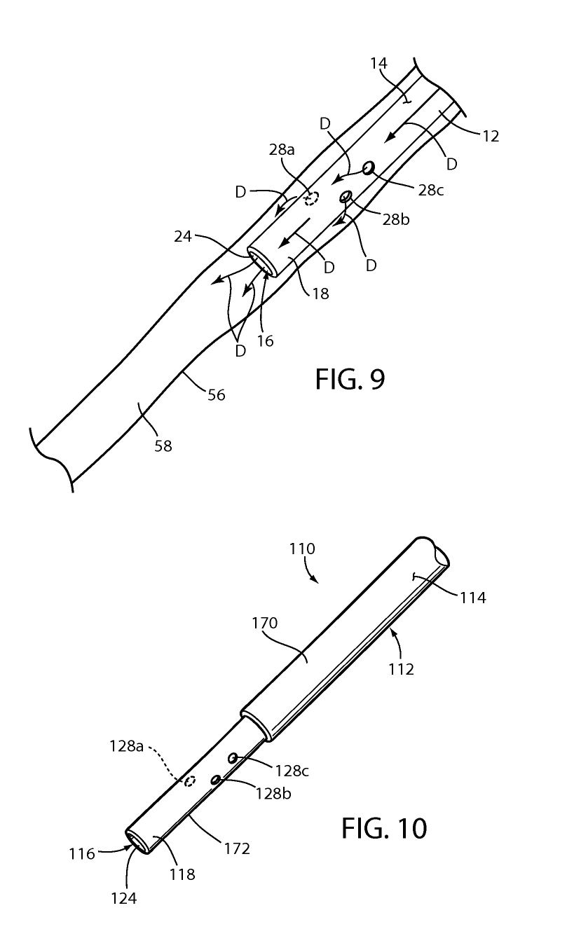

Returning to FIGS. 1-4, sheath 12 is illustrated as being coupled with a port body 80 that is coupled over second end 20 of sheath 12 and which is in communication with lumen 16 through access opening 26, which may both be positioned within port body 80. Port body 80 can include an infusion port 82 as well as a device port 84 therein that are both in communication with access opening 26 of sheath 12 such that guidewire can extend out of access opening 26 and further out of port body 80 through device port 84 to allow for catheter 78 to assembled thereover and guided into lumen 16 through device port 84. Additionally, infusion port 82 can be provided to connect with a delivery tube 86 to provide various substances, including liquid medication or the like to lumen 16 of sheath body 12. As shown in FIG. 9, such medication can be instantaneously forced under pressure through lumen 16 to provide localized delivery for such medication, which can occur through both vessel opening 24 and through perfusion windows 28, as indicated by delivery path lines d in FIG. 9. In another example, device 10 can be similarly used to achieve antegrade access to a similar vessel of a limb of a patient.

As shown in FIG. 10, an alternative embodiment of device 110 can include a sheath 112 that includes both a main body portion 170 and an extension body 172 that can be made to be telescoping with respect to main body 170. In such an embodiment, window area 162 can be positioned along extension body 172 such that perfusion windows 128a, 128b, and 128c are positioned on extension body 172. Such an embodiment 110 can provide for an adjustable length of sheath 112 to achieve positioning of vessel opening 124 as well as perfusion windows 128 at a desired distance from an insertion point of sheath 112. Sheath 112 can be inserted into a vascular (e.g., arterial) lumen of a patient in a manner similar to sheath 12, discussed above, and can further be used to provide access to the vasculature of a patient in a similar manner to sheath 12, including the provision of a perfusion path for blood flow between vessel opening 24 and perfusion windows 128.

It will be understood by one having ordinary skill in the art that construction of the described invention and other components is not limited to any specific material. Other exemplary embodiments of the invention disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the invention as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present invention. The exemplary structures and processes disclosed herein are for illustrative purposes and are not to be construed as limiting.

1. A vascular access device, comprising:
a sheath body defining an outer surface and a lumen extending between a first end of the sheath body and a second end of the sheath body, wherein:
the sheath body has a length of at most 7 cm;
the sheath body has a vessel opening defined through the first end in communication with the lumen and an access opening defined through the second end in communication with the lumen;
a plurality of perfusion windows extend between and are open to the lumen and the outer surface of the sheath body.

2. The vascular access device of claim 1, wherein the plurality of perfusion windows includes exactly three perfusion windows, each of the plurality of perfusion windows being located within about 3 cm of the first end of the sheath body.

3. The vascular access device of claim 1, wherein the plurality of perfusion windows are distributed along a section of the length of the sheath body and around a circumference of the outer surface of the sheath body in a spiral pattern.

4. The vascular access device of claim 3, wherein the section of the length of the sheath body along which the perfusion windows are distributed is between about 1 cm and about 2 cm in length.

5. The vascular access device of claim 3, wherein the section of the length of the sheath body along which the perfusion windows are distributed is spaced apart from the first end of the sheath body by between about 1 cm and 2 cm.

6. The vascular access device of claim 3, wherein the plurality of perfusion windows is distributed along the section of length of the sheath body so as to be spaced apart from each other by between about 5 mm and 10 mm.

7. The vascular access device of claim 1, wherein each of the plurality of perfusion windows has a diameter of between about 8/1000 in and about 12/1000 in.

8. The vascular access device of claim 1, wherein the lumen has a cross-sectional diameter of about 2.9 fr.

9. The vascular access device of claim 1, further including a port body connected with the sheath body over the second end thereof and including an infusion port and in fluid communication with the lumen.

10. The vascular access device of claim 1, wherein the sheath body includes a main body portion and an extension body portion coupled with the main body portion so as to be extendable therefrom and at least partially retractable thereinto, the perfusion windows being defined on the extension body portion.

11. A vascular access system, comprising:
an access sheath including a body extending between a first end and a second end, having a length of at most 7 cm, and defining a lumen therethrough open between the first end at a vessel opening and the second end at an access opening, an outer surface of the access sheath extending between the first end and the second end coaxially with the lumen, and a plurality of perfusion windows extending between and open to the lumen and the outer surface of the sheath body; and
an introducer assemblable within the lumen of the access sheath through the access opening thereof so as to extend outwardly through the vessel opening.

12. The system of claim 11, wherein:
each of the plurality of perfusion windows is positioned within about 3 cm of the first end of the sheath body.

13. The system of claim 11, wherein the introducer defines a tip portion that extends outwardly beyond the first end of the sheath when the introducer is assembled with the access sheath, the introducer defining an outer surface that is tapered within the tip portion from a first diameter that substantially matches a diameter of the lumen to a second diameter that is less than the first diameter.

14. The system of claim 13, wherein:
the outer surface of the access sheath tapers within a tapered portion adjacent the first end from a first diameter to a second diameter smaller than the first diameter at the first end of the sheath body; and
the tip portion of the introducer substantially aligns with the outer surface of the access sheath within the tapered portion thereof.

15. The vascular access device of claim 13, wherein:
the diameter of the lumen is about 2.9 fr;
the tip portion of the introducer has a length of at least 2 cm; and
the second diameter of the tip of the introducer is about 21/1000 in.

16. The system of claim 11, wherein:
the introducer defines a guidewire path therethrough; and
the system further includes a guide wire receivable through the guidewire path when the introducer is assembled within the lumen of the access sheath.

17. The system of claim 11, further including a catheter receivable through the lumen of the access sheath in place of the introducer and extendable out from the subcutaneous end by a distance of at least 10 cm.

18. The system of claim 17, wherein the catheter is undersized relative to the lumen of the access sheath such that at least one fluid perfusion path is present between the lumen at the vessel opening and the plurality of perfusion windows.

19. A vascular access sheath, comprising:
a sheath body defining an outer surface and an interior lumen, the sheath body defining a first end and a second end opposite the first end, the outer surface and the interior lumen extending coaxially therebetween, a plurality of perfusion windows extending between and open to the lumen and the outer surface of the sheath body, all of such perfusion windows being positioned within about 3 cm of the first end;
wherein the lumen defines:
an access path between an access opening through the second end and a vessel opening through the first end; and
a perfusion path between the vessel opening and the perfusion windows.

20. The vascular access sheath of claim 19, wherein the perfusion path is generally defined around at least a portion of the access path.
